(19) United States
(12) Patent Application Publication
(10) Pub. No.: US 2006/0227939 A1
(43) Pub. Date: Oct. 12, 2006

(54) Bite piece for a dental x-ray system

(75) Inventors: Donald Walker, Mundelein, IL (US); David Pettinato, Schaumburg, IL (US)

Correspondence Address: MCNEES, WALLACE & NURICK LLC, 100 PINE STREET, P.O. BOX 1166, HARRISBURG, PA, 17108-1166, US

(73) Assignee: GENDEX CORPORATION, Washington, DC (US)

(21) Appl. No.: 11/103,026

(22) Filed: Apr. 11, 2005

(51) Int. Cl. H05G 1/00; A61B 6/14

(52) U.S. Cl. 378/208; 378/38

(57) ABSTRACT

A bite piece is provided for use with a dental x-ray system. A substantially straight first portion extends to a curved second portion. An end of the second portion opposite the first portion has a region of reduced cross section adjacent the end of the second portion. The region is received between a patient's opposed upper and lower anterior teeth to secure the patient's head position during operation of the dental x-ray system. The region and the second portion substantially lack transitional edges, improving each of the quality of a radiograph produced during operation of the dental x-ray system, a patient's level of comfort and stability of the patient's head position.

### FIELD OF THE INVENTION

The present invention is directed to a bite piece construction for use with dental equipment, and more particularly, is directed to a bite piece construction that is used in dental x-ray systems.

### BACKGROUND OF THE INVENTION

X-ray systems are commonly used in the dental industry to assist dental professionals. One type of a dental x-ray system uses a diagnostic panoramic technique to take a contiguous radiograph of the entire dentition of the jaw. The panoramic radiograph is achieved by positioning the patient between the x-ray source and a receiving radiosensitive surface. The x-ray source and receiving radiosensitive surface are maintained at a predetermined relationship from each other and revolved around the patient's head while the x-ray beam is continuously directed at the patient.

The x-ray source and receiving radiosensitive surface rotate in a path about a vertical axis and create a vertically orientated focal trough. The focal trough is positioned with relation to the patient's chin resting on a chinrest. Once the patient's chin position has been established, the x-ray system operator must ensure the patient's head is oriented correctly. To position the patient's head at a desired horizontal position, the patient's Frankfort plane is positioned horizontally. The Frankfort Plane is an imaginary line passing through the external ear canal and across the top of the lower bone of the eye socket, immediately under the eye. Similarly, the patient's head must be positioned at a desired vertical position, which is achieved by centering the patient's midsagittal plane vertically, the sagittal plane defining a longitudinal vertical plane that divides the mouth into two halves (left and right).

Failure to achieve correct vertical positioning results in the dental arch passing through the dental trough at an angle in the anterior region. The resulting image will be distorted in the anterior portion of the film through the maxillary and mandibular regions, causing foreshortening of the root tips, superimposition of the hard palate across the maxillary root tips, as well as blurriness to the dentition that extends beyond the edges of the focal trough. Failure to achieve correct vertical positioning also results in the dental arch not being laterally positioned (centered) within the focal trough. If the patient's head is not centered within the focal trough, one side of the arch will be closer and the other side of the arch will be farther from the film. This non-centered condition will result in an increase in magnification on one side of the image and a decrease in magnification on the other side. An additional negative result is an image containing an increase in the amount of inherent overlapping of the teeth due to the x-ray beam not passing perpendicularly through the dental arch.

A bite piece, also referred to as a bite block, is used to assist the operator to properly position the patient within the focal trough. This is achieved by having the patient bite down on the bite piece while resting her chin on the chinrest. However, conventional bite piece constructions can be large, cumbersome devices that are uncomfortable to use. Additionally, conventional bite pieces often have edges or abrupt changes in cross sectional profile which can obscure and significantly lessen the clarity, and therefore the quality of the radiograph.

What is needed is a bite piece for use with a dental x-ray device that is inexpensive to manufacture, comfortable to use, provides stability to secure the patient's head position and does not obscure the resulting radiograph.

### SUMMARY OF THE INVENTION

The present invention relates to a bite piece for use with a dental x-ray system including a first portion extending to a second portion. An end of the second portion opposite the first portion has a region of reduced cross section adjacent the end, the region configured and disposed to be received between a patient's opposed upper and lower anterior teeth to secure the patient's head position during operation of the dental x-ray system, the region and the second portion substantially lacking a transitional edge.

The present invention further relates to a chin rest for use with a dental x-ray system including a body having a recess formed therein to secure a patient's chin thereon, the body having a slot formed therein adjacent the recess. A first portion extends to a second portion, the first portion being slidably received in the slot. An end of the second portion opposite the first portion has a region of reduced cross section adjacent the end, the region configured and disposed to be received between a patient's opposed upper and lower anterior teeth to secure the patient's head position during operation of the dental x-ray system, the region and the second portion substantially lacking a transitional edge.

An advantage of the present invention is that it can be inexpensively manufactured.

A further advantage of the present invention is that it provides a large lateral biting surface to provide improved stability.

A yet additional advantage is that the large lateral biting surface is of reduced thickness to provide improved patient comfort.

A still further advantage of the present invention is that it has a bite groove and bulb end profile angled away from the teeth bilaterally to alleviate patient teeth and gum discomfort associated with the patient biting on the groove.

An additional advantage of the present invention is it minimizes the erroneous projection of artifacts onto the radiosensitive film during operation of the x-ray system.

A further advantage of the present invention is that it reduces the amount of shadowing projected onto the radiosensitive film during operation of the x-ray system.

An additional advantage of the present invention is that it provides improved radiograph quality due to the contour of the bite piece and increased patient head position stability.

A still yet further advantage of the present invention is that it secures the patient's head position.

Other features and advantages of the present invention will be apparent from the following more detailed description of the preferred embodiment, taken in conjunction with the accompanying drawings which illustrate, by way of example, the principles of the invention.

### BRIEF DESCRIPTION OF THE DRAWINGS

FIG. 1 is a perspective view of a dental apparatus having a bite piece of the present invention.

FIG. 2 is a perspective view of a chinrest having a bite piece of the present invention.

FIG. 3 is an elevation view of a bite piece of the present invention.

FIG. 4 is a perspective view of a bite piece of the present invention.

FIG. 5 is a cross section of an embodiment of a sliding portion of a bite piece of the present invention.

FIG. 6 is a cross section of an embodiment of a groove taken along line 6-6 of FIG. 3 of a bite piece of the present invention.

FIGS. 7-8 are perspective views of articulating wands for use with a chinrest of a dental apparatus of the present invention.

FIGS. 9-10 are cross sections of a chinrest taken along line 9-9 of FIG. 8 of a dental apparatus of the present invention.

FIG. 11 is a cross section of an embodiment of a groove taken along line 6-6 of FIG. 3 of a bite piece of the present invention.

FIG. 12 is a partial exploded view of a mechanical linkage for a chinrest of a dental apparatus of the present invention.

Wherever possible, the same reference numbers will be used throughout the drawings to refer to the same or like parts.

### DETAILED DESCRIPTION OF THE INVENTION

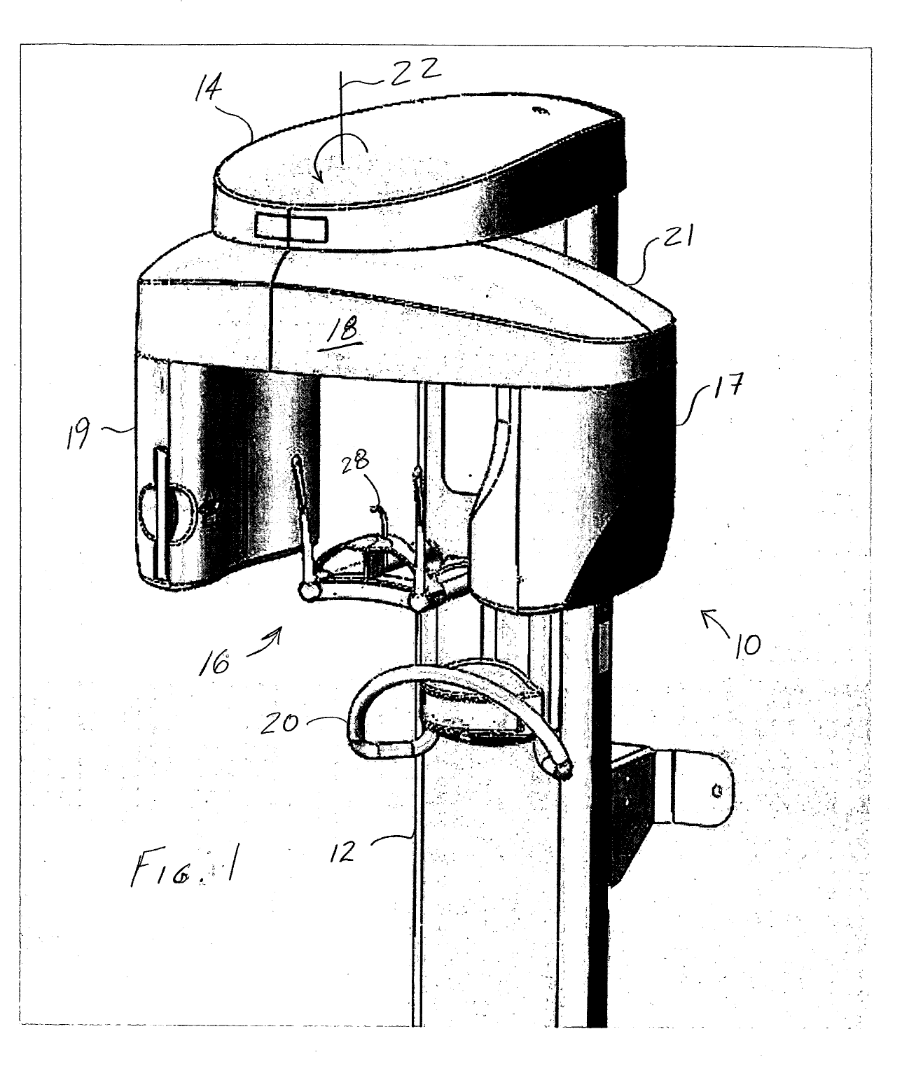

One embodiment of a dental apparatus 10 having a bite piece 28 of the present invention is depicted in FIG. 1. Preferably, the dental apparatus 10 includes a column 12 that extends from the floor supporting the dental apparatus 10, the column 12 slidingly supporting a rail 20 and a chinrest 16. Rail 20 can include one or more bars extending away from column 12 in a direction that is ergonomically friendly for a patient (not shown) to grasp while the dental apparatus 10 is in operation. The rail 20 permits the patient (not shown) to conveniently stabilize her position once the patient's chin has been placed and positioned on the chinrest 16. A cantilevered member 14, which extends from the end of the column 12 opposite the floor, rotatably carries an inverted U-shaped structure 18 about a substantially vertical axis 22. The U-shaped structure 18 includes a first projection 17 containing an x-ray source and a second projection 19 containing a radiosensitive material, the projections 17, 19 being connected to and supported by a web 21. During operation of the dental apparatus 10, the projections 17, 19 are rotated about the head of the patient, producing a contiguous radiograph of the entire dentition of the patient's jaw.

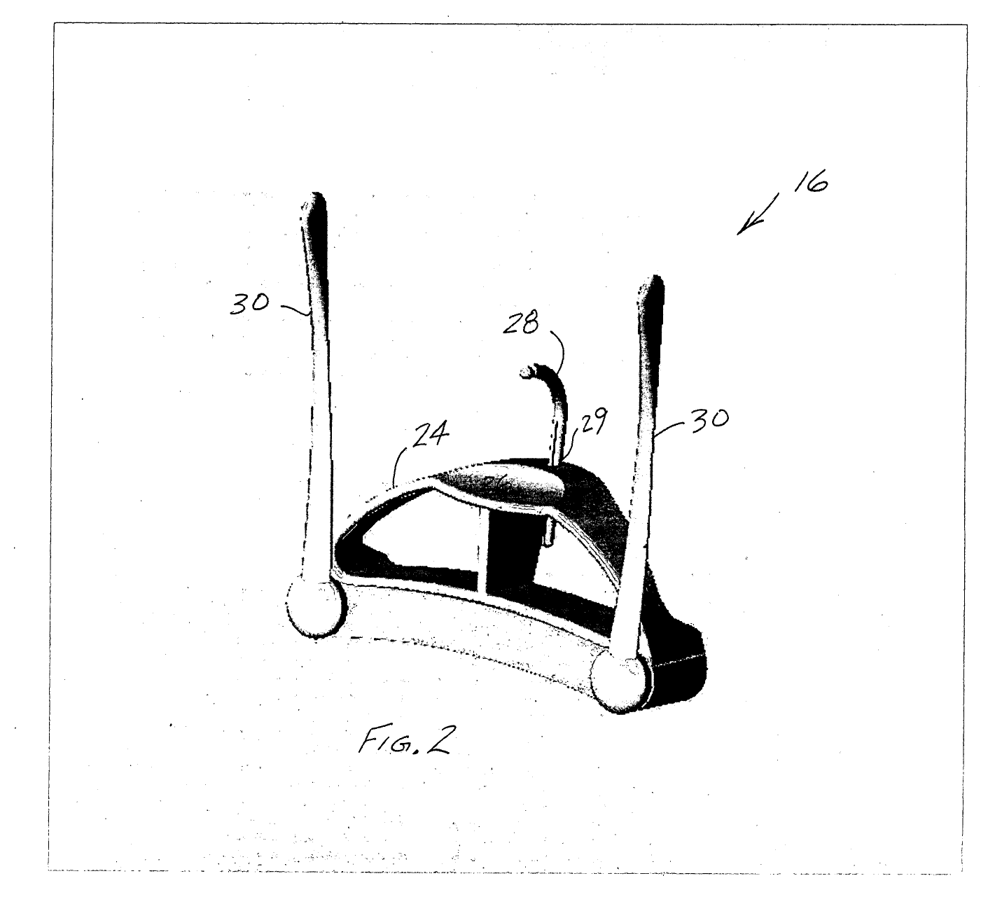

Referring to FIGS. 1-4, the chinrest 16 includes a body 24 having a recess 26 for securing a patient's chin thereon and a slot 29 for slidably receiving the bite piece 28. Preferably, the patient's upper and lower anterior teeth are brought together in a circumferential groove 44 formed in the bite piece 28 to secure the patient's head in a fixed position during operation of the dental apparatus 10. Optionally, the groove 44 is not circumferential, as further discussed below. To help prevent inadvertent lateral rotation of the patient's head during operation of the dental apparatus 10, a pair of arms 30 hingedly mounted to opposed ends of the body 24 are rotated into contact with the patient's head. However, other constructions to contact the head can also be used, including, for example, a device protruding from the U-shaped structure. Preferably, the position of the chinrest 16 and rail 20, as well as the U-shaped structure 18 containing the x-ray source and radiosensitive film can be adjusted to accommodate the patient.

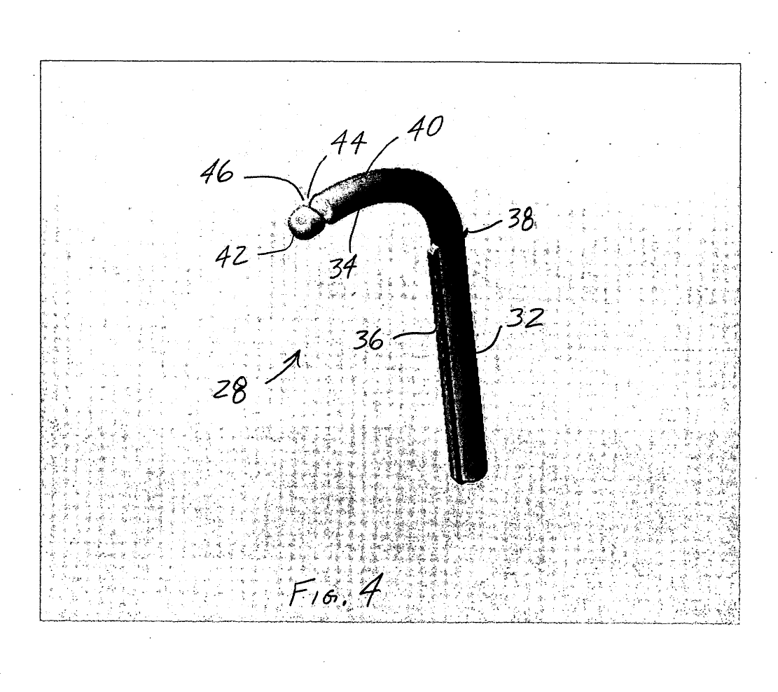

Referring to FIGS. 1-6, the bite piece 28 includes a sliding portion 32 that extends to a curved portion 34. The sliding portion 32 is preferably substantially straight, terminating at a curved portion 34, the sliding portion 32 being received by the slot 29 in the chinrest 16. The sliding portion 32 preferably has a D-shaped cross section, and more preferably, substantially opposite the flat portion of the D-shape, a rib 36 extends radially outward from the sliding portion 32. The D-shaped cross section and rib 36 which are received in the corresponding slot 29 in the chinrest 16, helps to prevent the bite piece 28 from rotating about the axis of the sliding portion 32 within the slot 29. By preventing such movement, the position of the patient's head is further stabilized. Preferably opposite the rib 36 and separating the curved portion 34 from the sliding portion 32, a stop 38 extends outwardly from the bite piece 28 to provide a minimum height position that the curved portion 34 extends above the chinrest 16. While a D-shaped cross section is shown for the sliding portion 36, it is to be understood that other shapes may also be used, such as circular, hex, square, etc.

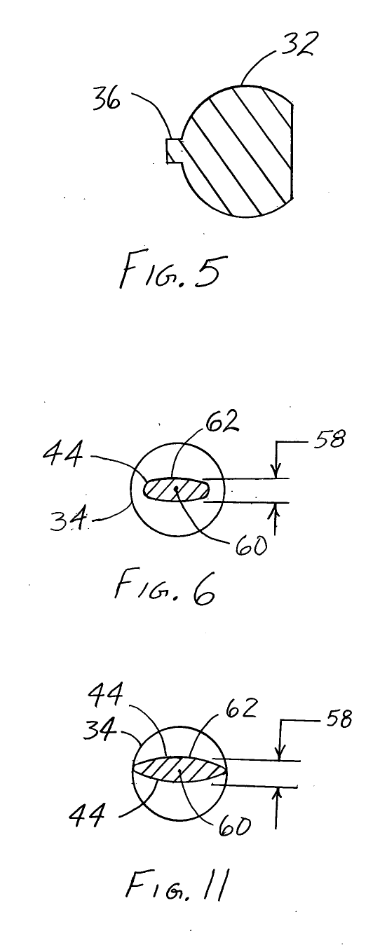

The curved portion 34 preferably defines a curved path extending from the stop 38 at one end to a bulb 42 at the other end. Preferably, the degree of curvature of the curved portion 34 adjacent to the stop 38 is more pronounced, gradually extending to a substantially straight portion adjacent to the bulb 42. Preferably, the substantially straight portion is substantially horizontal for ease of access by a patient. To further provide improved patient access, circumferential groove 44 is formed adjacent to the bulb 42 having clearance regions 46 between the bulb 42 and the curved portion 34. It is intended that the patient bites down on the groove 44, the patient's upper and lower anterior teeth, or incisors, contacting the opposed sides of the groove 44. In a preferred embodiment, a reduced distance 58 between opposed sides of the groove 44 is shown in FIG. 6 by intersecting the groove 44 with a substantially vertical plane 48, the intersection forming a substantially elliptical cross section 62. Preferably, the plane 48 is perpendicular to an axis 60 that extends along the length of the bulb 42 through the center of the bulb 42. The elliptical cross section 62 provides increased stability to secure the patient's head position due to the increased surface contact with the patient's teeth, versus other geometric arrangements, such as a circular cross section. In addition to enhanced patient positioning stability, the patient comfort level is increased, due to the reduced distance between opposed sides of groove 44.

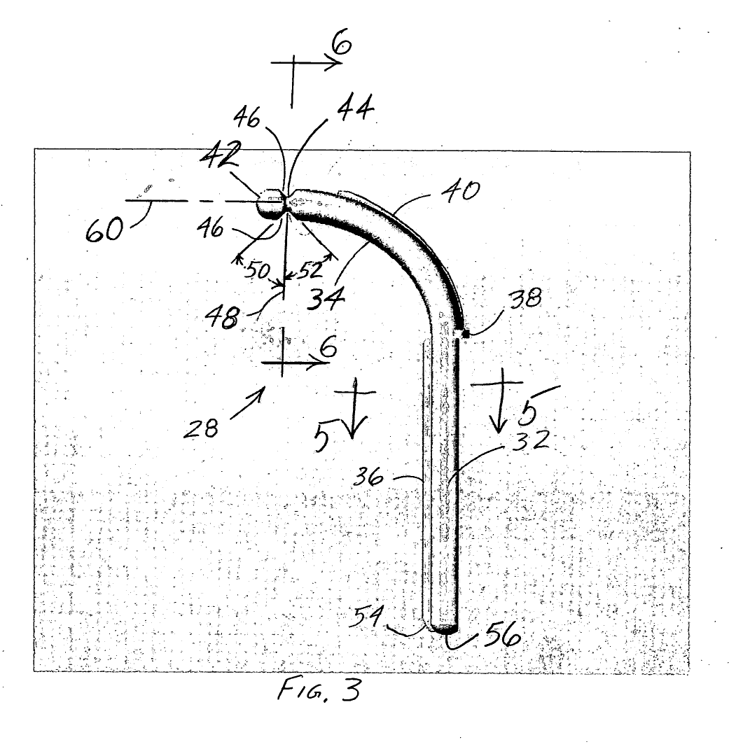

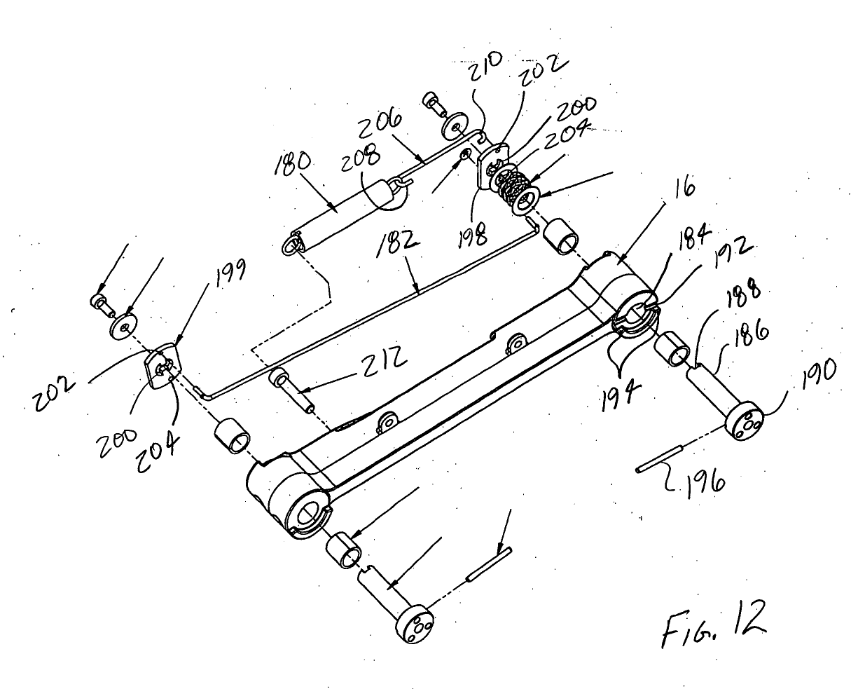

Optionally, groove 44 is not circumferential. That is, as shown in FIG. 11, which is a cross section taken along line 6-6 of FIG. 3, similar to FIG. 6, the elliptical cross section 62 extends laterally to the opposed portions of the outer surface of the juncture between the bulb 42 and the curved portion 34. The groove 44 is then bifurcated into upper and lower portions or sections that are otherwise similar to the embodiment of the groove 44 as shown in FIG. 6. However, irrespective the embodiment, it is to be understood that while the reduced distance 58 is preferably substantially horizontally centered within the cross sections as shown in FIGS. 6 and 11, this is not required. Stated another way, the maximum depth of the groove 44 along the upper portion is not necessarily equal to the maximum depth of the groove 44 along the lower portion.

Further increasing patient comfort is the profile of the clearance region 46 between the bulb 42 and the curved portion 34. Preferably, the vertical plane 48 defines a clearance angle 50 between the vertical plane 48 and the adjacent surface of the bulb 42. Similarly, a clearance angle 52 is defined between the vertical plane 48 and the adjacent surface of the curved portion 34. In a preferred embodiment, clearance angles 50 and 52 are substantially equal, and the transitions between clearance angles 50 and 52 and adjacent surfaces of the curved portion 34 or bulb 42 are rounded. It is also preferred that clearance angles 50, 52 correspond to both the lower surface of the groove 44 and the upper surface of the groove 44. Clearance angles 50, 52 are configured to provide sufficient clearance between the patient's teeth and gums and the adjacent surfaces of the bulb 42 and curved portion 34. Preferably, the clearance angles 50, 52 can range from about 20 to about 50 degrees. It is to be understood that while clearance angles are shown between portions of the groove 44 and adjacent surfaces of the bulb 42 and curved portion 34, clearance regions 46 can be defined by any profile obtained by removing material between the bulb 42 and the curved portion 34 to form the resulting groove 44 which does not produce an edge that would cause discomfort to the patient.

The bite piece further includes a ridge 40 that extends outwardly from the curved portion 34 to assist in patient head alignment. The ridge 40, which is preferably located along the centerline of the upper surface of the curved portion 34, establishes the patient's midsaggital plane to properly align the patient laterally within the focal trough. In other words, the ridge 40 can be used as a visual alignment reference in conjunction with alignment features which can be a part of the x-ray system, such as a laser generated midsaggital line that is projected from the x-ray system.

While the cross sectional profiles of the curved portion 34 and bulb 42 define rounded cross sectional shapes, importantly, the rounded cross sectional shapes are substantially absent of sharp transitional edges. Since such transitional edges can introduce erroneous artifacts in the radiograph, the quality of the radiograph is improved. Further, in a preferred embodiment in which the cross sectional profile of the curved portion 34 and bulb 42 is elliptical, the quality of the radiograph is still further enhanced. This additional enhancement is due to the reduced cross sectional area of material through which the x-ray beam must pass. By reducing the amount of cross sectional area of the curved portion 34, including the bulb 42 and the groove 44, the amount of shadowing on the resulting radiograph is reduced, thereby increasing clarity of the radiograph.

Although the bite piece 28 can be constructed of any material compatible with x-ray exposure and patient use having sufficient material strength and stiffness, it is preferred that a polymer such as high density polyethylene (HDPE) is used. HDPE is fully recyclable and disposable. In addition, since HDPE is incapable of withstanding the temperatures associated with autoclaving, constructing the bite piece of HDPE restricts use of the bite piece 28 to a single patient.

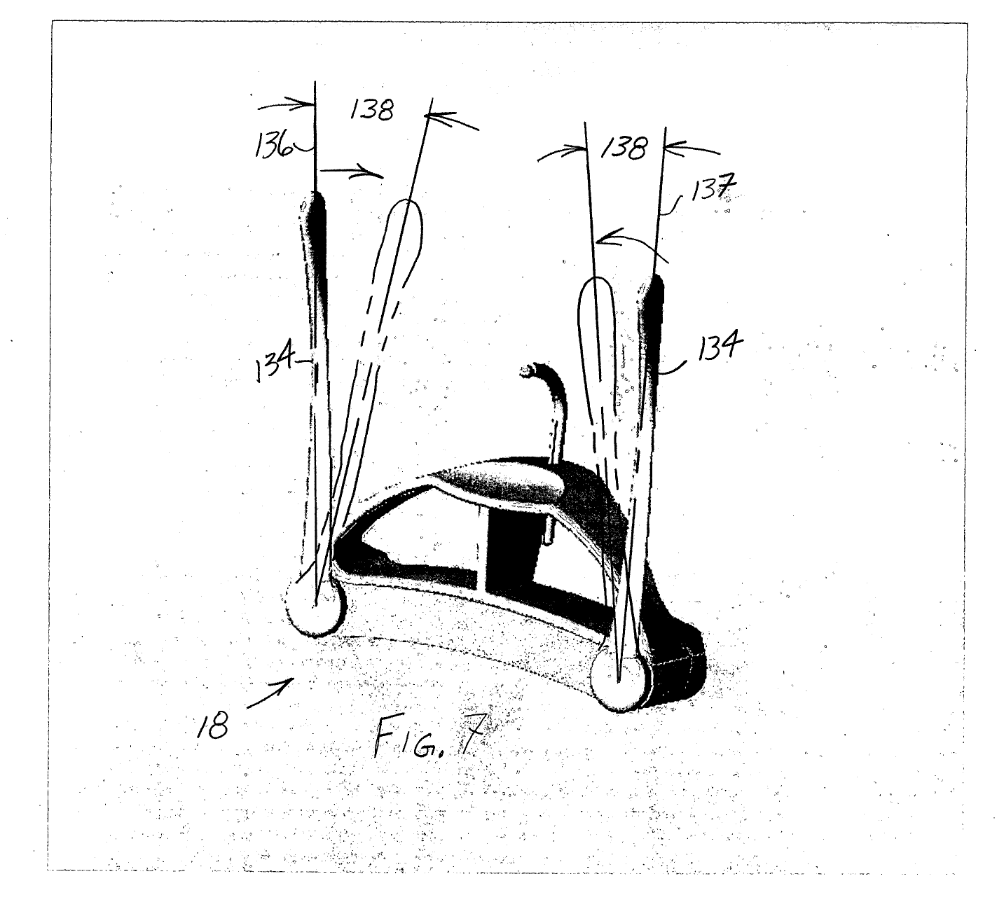

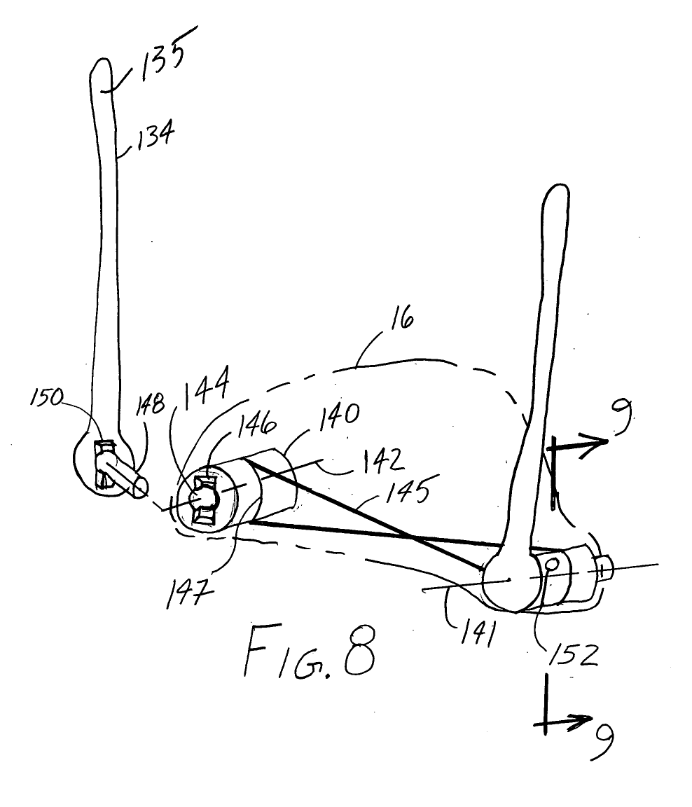

Referring to FIGS. 7-10 is an embodiment of a pair of adjustable temple support wands 134 for use with the chinrest 16. Wand 134 is an elongate member extending from a substantially flat portion 135 at one end for contacting a patient's head that has been positioned on the chinrest 16. The other end of the wand 134 extends to a projection portion 150 having a noncircular profile, further extending to a shaft portion 148 that preferably defines a circular profile. Housed within the body of the chinrest 16 are a pair of cylinders 140 each rotatable within the chinrest 16 about a respective axis 141, 142. Each cylinder 140 includes a passageway 144 having a noncircular recess portion 146 adjacent the opening of the passageway 144. Preferably, the passageway 144 receives the shaft portion 148 and the noncircular recess portion 146 receives the noncircular projection portion 150. By virtue of the meshing of the noncircular recess portion 146 with the noncircular projection portion 150, each wand 134 rotates in unison with the cylinder 140 about respective axis 141, 142.

To control the relative rotational movement between the wands 134, a connecting member, such as a belt 145, is in simultaneous frictional contact with a portion of each of the cylinders 140, such as by extending around each cylinder 140 within a groove 147. The tension in belt 145 is sufficient to provide the desired frictional contact. By crossing the belt 145 over itself to form a figure eight configuration, one having skill in the art can appreciate that rotating one wand 134 an angle 138 away from a reference vertical axis 136 toward the other wand 134 similarly urges the other wand 134 to rotate toward the one wand 134 from its reference vertical axis 137. Reference vertical axes 136 and 137 are substantially parallel. Stated another way, wands 134 are configured to rotate at equal angles toward each other by virtue of frictional contact with the belt 145. By achieving symmetric rotation with respect to the vertical axes 136, 137, the respective ends 135 of each wand 134 are brought toward each other to orient a patient's head in a centered position, thereby achieving a consistent, repeatable positioning system. In other words, upon the end 135 of each wand 134 contacting opposite sides of a patient's head, the patient's head is secured in a centered position to provide an optimal, repeatable position for obtaining a dental radiograph. A small amount of compressive force is desirable to maintain the wand ends 135 in contact with the patient's head. This can be easily achieved with a torsion spring (not shown) or other similar device. That is, the torsion device urges the ends 135 of the wand 134 toward each other.

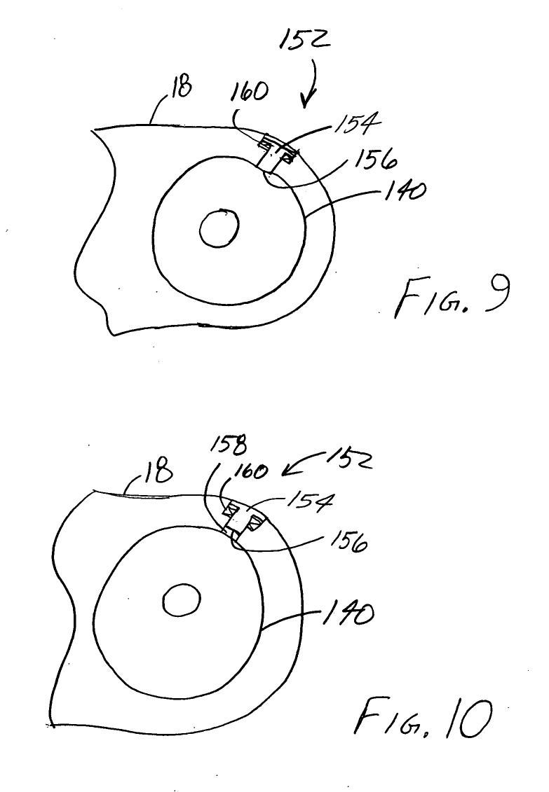

While the compressive force of the ends 135 of the wands 134 toward each other is desirable once the patient's head is positioned on the chinrest 16 and the dental apparatus 10 is operating, this is not always the case. In other words, when the patient's is initially being positioned on the chinrest 16, it is not yet desirable that the ends 135 of the wand 134 are positioned adjacent to each other, as the patient's head must first be positioned between the wands. It is desirable to have a detent, such as a spring loaded plunger 154 to lock the rotational orientation of the wands 134 in a position wherein the ends 135 are sufficiently spaced so that the patient can place her head between the wands 134. Once the patient has positioned her head on the chinrest 16, the detent can then be placed in an unlocked position, permitting the ends 135 to rotate toward each other to secure the patient's head as previously discussed. FIG. 9 shows the locked position of the detent 152 which is achieved by depressing the head of the plunger 154 that is locked in its depressed position by a locking device (not shown) so that the tip 156 of the plunger 154 is placed in abutting contact with the outer surface of the cylinder 140, preventing the cylinder 140 from rotating. Conversely, FIG. 10 shows the unlocked position of the detent 152 which is achieved by again depressing the head of the plunger 154 to disengage the locking device. Disengaging the locking device permits a spring 160 that is disposed between the body of the chinrest 16 and the head of the plunger 154 to urge the tip 156 of the plunger 154 out of contact with the surface of the cylinder 140, defining a gap 158 therebetween. By forming the gap 158 between the plunger 154 and the cylinder 140, the torsion spring then urges the ends 135 of the wand 134 toward each other to contact the patient's head. Therefore, the detent 152 provides a convenient way to maintain the wands 134 out of the way of the patient until use of the wands 134 is desired, and then activating the wands 134 by pressing the detent 152.

Alternately, FIG. 12 shows an alternate arrangement to achieve the desired movement of the wands 134. The chinrest 16 includes a pair of passageways 184 each receiving a shaft portion 186 that is then connected to a wand 134. The shaft portion 186 has a notched portion 188 at one end and a head 190 at the other end that is connected to the wand 134. A pin 196 is inserted adjacent to the head 190. Each passageway 184 includes a set of stops 194 for limiting the rotation of the shaft portion 186 in each direction due to the pin 196 abutting against the stops 194. The notch 188 of the shaft portion 186 engages a notched aperture 200 of a plate 198 having opposed apertures 202, 204. A spring 180 is secured at one end by a pin 212 and a hook 208 of a linking member 206 at the other end. A hook 210 of the linking member 206 that is opposite the hook 208 engages aperture 202 of the plate 198. One end of a connecting rod 182 engages aperture 204, the other end of the connecting rod 182 engaging aperture 202 of a plate 199.

The spring 180 and linking member 206 are sized to urge the aperture 202 of plate 198 into rotation toward plate 199. Similarly, by virtue of the connecting rod 182, the aperture 204 of plate 199 is urged into rotation toward plate 198. Further, the rotations of plates 198, 199 (and wands 134) are substantially equal and opposite as that previously discussed. However, the detent feature as shown in FIG. 12 operates differently than the detent previously discussed. One passageway 184 includes a groove 192 formed adjacent the stops 194. In normal operation, the pin 196 rotates about the axis of shaft portion 186 without encountering the groove 192. To make use of the detent feature, which locks the wands 134 in a fixed position, a force is applied to the head 190 of the shaft portion 186 directing the head 190 toward the spring 180. Simultaneously, the head 190 is rotated to a predetermined position so that the groove 192 and the pin 196 are aligned. Once the alignment between the pin 196 and the groove 192 is achieved, the pin 196 is engaged in the groove 192, thereby achieving the detent lock. To unlock the detent, sufficient force applied to the head 196 away from the spring 180 disengages the pin 196 from the groove 192.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A bite piece for use with a dental x-ray system comprising:
a first portion extending to a second portion; and
an end of the second portion opposite the first portion having a region of reduced cross section adjacent the end, the region configured and disposed to be received between a patient's opposed upper and lower anterior teeth to secure the patient's head position during operation of the dental x-ray system, the region and the second portion substantially lacking a transitional edge.

2. The bite piece of claim 1 wherein the region is substantially elliptical.

3. The bite piece of claim 1 wherein the second portion defines a bulb.

4. The bite piece of claim 1 wherein at least one groove is formed adjacent the region.

5. The bite piece of claim 4 wherein the at least one groove is circumferential.

6. The bite piece of claim 4 wherein the at least one groove defines at least one clearance angle between the first portion and the second portion.

7. The bite piece of claim 6 wherein the at least one clearance angle is between about 20 degrees and about 50 degrees.

8. The bite piece of claim 4 wherein the at least one groove defines at least one clearance angle along at least one of an upper section and a lower section between the first portion and the second portion.

9. The bite piece of claim 1 wherein the bite piece is a polymer.

10. The bite piece of claim 9 wherein the polymer is recyclable and disposable.

11. The bite piece of claim 10 wherein the polymer is incapable of withstanding the temperatures associated with autoclaving.

12. A chinrest for use with a dental x-ray system comprising:
a body having a recess formed therein to secure a patient's chin thereon, the body having a slot formed therein adjacent the recess;
a first portion extending to a second portion, the first portion being slidably received in the slot; and
an end of the second portion opposite the first portion having a region of reduced cross section adjacent the end, the region configured and disposed to be received between a patient's opposed upper and lower anterior teeth to secure the patient's head position during operation of the dental x-ray system, the region and the second portion substantially lacking a transitional edge.

13. The chinrest of claim 12 wherein the region is substantially elliptical.

14. The chinrest of claim 12 wherein the second portion defines a bulb.

15. The chinrest of claim 12 wherein at least one groove is formed adjacent the region.

16. The chinrest of claim 15 wherein the at least one groove is circumferential.

17. The chinrest of claim 15 wherein the at least one groove defines at least one clearance angle between the first portion and the second portion.

18. The chinrest of claim 17 wherein the at least one clearance angle is between about 20 degrees and about 50 degrees.

19. The chinrest of claim 15 wherein the at least one groove defines at least one clearance angle along at least one of an upper section and a lower section between the first portion and the second portion.

20. The chinrest of claim 12 wherein the first portion includes an outwardly extending rib.

21. The chinrest of claim 12 wherein the first portion includes an outwardly extending stop to provide a minimum height position of the second portion above the body.

22. The chinrest of claim 12 comprises a first member and a second member each having a first end and a second end opposite the first end, the first ends being rotatably carried by the body, the second ends being configured to contact a patient's head positioned on the chinrest.

23. The chinrest of claim 20 wherein first and second members are symmetrically rotatable about respective first ends to orient a patient's head in a centered position.

24. The chinrest of claim 20 comprises a detent disposed in the body to secure the first and second members in a predetermined orientation.
